(19) United States
(12) Patent Application Publication
(10) Pub. No.: US 2018/0224508 A1
(43) Pub. Date: Aug. 9, 2018

(54) ELECTROMAGNETIC NAVIGATION SYSTEM HAVING A DEMODULATION UNIT

(71) Applicant: Boston Scientific Scimed Inc., Maple Grove, MN (US)

(72) Inventors: David W. Kelly, Eagan, MN (US); Eric P. Bailey, Roseville, MN (US)

(21) Appl. No.: 15/888,969

(22) Filed: Feb. 5, 2018

Related U.S. Application Data
(60) Provisional application No. 62/455,281, filed on Feb. 6, 2017.

(51) Int. Cl. G01R 33/00; H04J 1/12; H04J 11/00; G01D 5/14; A61B 5/06

(52) U.S. Cl. CPC G01R 33/0029; H04J 1/12; H04J 11/0063; G01R 33/02; A61B 5/062; H04J 2011/0096; G01D 5/14

(57) ABSTRACT

A method uses an electromagnetic navigation system, including a number of field transmitters and at least one receiver. The method includes receiving a field signal from the at least one receiver, and the field signal includes a number of different frequencies corresponding to each of the field transmitters. The received field signal is multiplied by a synchronization signal using a frequency-division multiplexing scheme, and the received field signal is integrated over a predetermined sampling interval and converted into an integrated signal having only a desired signal based on the synchronization signal. The integrated signal is outputted for subsequent processing of the electromagnetic navigation system.

### CROSS-REFERENCE TO RELATED APPLICATION

This application claims priority to Provisional Application No. 62/455,281, filed Feb. 6, 2017, which is herein incorporated by reference in its entirety.

### TECHNICAL FIELD

Embodiments of the present disclosure relate to electromagnetic navigation systems. More specifically, embodiments of the disclosure relate to signal processing in an electromagnetic navigation environment.

### BACKGROUND

Electromagnetic (EM) navigation systems are useful in medical device applications where the position and orientation of a medical device can often provide useful information in a minimally invasive or surgical medical procedure. Such systems often include a set of electromagnetic field generators (e.g., field transmitters) and one or more field sensors associated with a medical device that can measure signals from the set of transmitters. The measured signals are used with a computational algorithm to estimate the position and/or orientation of the medical device or a portion thereof (for example, the distal tip of a medical device).

A conventional EM navigation system includes multiple field transmitters, wherein each field transmitter generates a reference magnetic field with a specific frequency that is measured by the field sensors disposed at the distal tip of the medical device (e.g., a catheter). Typically, the measured signals from all field transmitters are combined and mixed together, causing signal interference and overlapping. Thus, the measured signal from each field transmitter must be separated and distinguishable from other measured signals. In procedures requiring a high degree of accuracy in position and/or orientation navigation, such field interference can result in errors in position or orientation that negatively impact the medical procedure. It is therefore desirable to seek methods to suppress or mitigate such errors due to interference in the environment of an electromagnetic navigation system.

While there have been previous attempts to address this problem, there is a need for a direct and simple-to-implement solution that does not place significant constraints on the hardware of a navigation system and does not result in significant additional computational demands. For example, conventional systems typically attempt to mitigate these types of errors by using time multiplexing and/or frequency multiplexing methods. The time multiplexing method enables one field transmitter at a time by limiting a field transmitter operating time, but reduces a signal-to-noise ratio (SNR), and provides less time for filtering out noise signals. In the frequency multiplexing method, each field transmitter broadcasts a different frequency. Each frequency associated with a particular field transmitter may be isolated by performing a cross correlation check between a field transmitter current and the measured signal, but this method provides slow response time and instability. As such, these approaches are computationally complex and inflexible due to calculations related to driving currents of the field transmitters and measured signals, further increasing computational costs.

### SUMMARY

Embodiments include methods and electromagnetic navigation systems for extracting a desired signal from a field signal received from a receiver. In embodiments, unwanted signals are suppressed by utilizing a demodulation process designed to select only the desired signal based on a synchronization signal using a frequency-division multiplexing scheme. In this manner, embodiments facilitate unwanted signal suppression by minimizing computational complexity and component counts since the received field signal is processed digitally for continuous frequency generation without distortive effects of noise and interference.

In an Example 1, a method for using an electromagnetic navigation system having a plurality of field transmitters and a receiver, the method comprising: receiving a field signal from the a receiver, the field signal having a plurality of different frequencies each corresponding to one of the plurality of field transmitters; multiplying the received field signal by a synchronization signal using a frequency-division multiplexing scheme; integrating the received field signal over a predetermined sampling interval and converting the received field signal into an integrated signal having only a desired signal based on the synchronization signal; and outputting the integrated signal for subsequent processing of the electromagnetic navigation system.

In an Example 2, the method of Example 1, further comprising suppressing unwanted signals having unselected frequencies from the received field signal.

In an Example 3, the method of either of Examples 1 or 2, further comprising amplifying the received field signal and converting the received field signal into a digital field signal.

In an Example 4, the method of any of Examples 1-3, further comprising selecting a predetermined frequency of the desired signal present in the received field signal.

In an Example 5, the method of any of Examples 1-4, further comprising filtering the received field signal by restricting a bandwidth of the received field signal.

In an Example 6, the method of any of Examples 1-5, further comprising downsampling the filtered received field signal by a predetermined integer factor.

In an Example 7, the method of any of Examples 1-6, further comprising correcting the received field signal by removing electrical crosstalk gained during the predetermined sampling interval from the received field signal.

In an Example 8, the method of any of Examples 1-7, further comprising: generating a first mixed signal based on the synchronization signal and the received field signal; and generating a second mixed signal based on the synchronization signal that is orthogonal and the received field signal.

In an Example 9, the method of Example 8, further comprising extracting only the desired signal based on the first mixed signal and the second mixed signal.

In an Example 10, the method of Example 8, further comprising filtering the first mixed signal based on a first predetermined cutoff frequency; and filtering the first mixed signal based on a second predetermined cutoff frequency.

In an Example 11, the method of any of Examples 1-10, wherein the frequency-division multiplexing scheme is an Orthogonal Frequency-Division Multiplexing (OFDM) technique.

In an Example 12, the method of any of Examples 1-11, wherein the synchronization signal is at least one of a clock signal, a sine signal, and a cosine signal.

In an Example 13, the method of any of Examples 1-12, wherein the received field signal is heterodyned with at least one of a sine signal and a cosine signal.

In an Example 14, the method of any of Examples 1-13, further comprising selecting transmission frequencies of the plurality of field transmitters such that heterodyne frequencies of the received field signals are orthogonal over a predetermined time period.

In an Example 15, the method of any of Examples 1-14, wherein the predetermined sampling interval is approximately 25 milliseconds with a measurement frequency of approximately 40 hertz.

In an Example 16, the method of any of Examples 1-15, further comprising generating a sum of zero integral value for unwanted signals having unselected frequencies from the received field signal based on the synchronization signal.

In an Example 17, an electromagnetic navigation system having a plurality of field transmitters and at least one receiver, the system comprising: a receiver configured for receiving a field signal from the at least one receiver, the field signal having a plurality of different frequencies corresponding to each of the plurality of field transmitters; a multiplier configured for selectively extracting a desired signal from the received field signal based on a synchronization signal and an orthogonal synchronization signal using a frequency-division multiplexing scheme; an integrator configured for integrating the received field signal over a predetermined sampling interval and converting the received field signal into an integrated signal having only a desired signal based on the synchronization signal; and an estimator configured for outputting the integrated signal for subsequent processing of the electromagnetic navigation system.

In an Example 18, the system of Example 17, further comprising: an amplifier configured for amplifying the received field signal; a converter configured for converting the received field signal into a digital field signal; a signal filter configured for filtering the received field signal by restricting a bandwidth of the received field signal; and a signal sampler configured for downsampling the filtered received field signal by a predetermined integer factor.

In an Example 19, the system of either of Examples 17 or 18, wherein unwanted signals of the received field signal are suppressed based on an orthogonal synchronization signal generated by the frequency-division multiplexing scheme.

In an Example 20, the system of any of Examples 17-19, wherein the frequency-division multiplexing scheme is an Orthogonal Frequency-Division Multiplexing (OFDM) technique.

While multiple embodiments are disclosed, still other embodiments of the presently disclosed subject matter will become apparent to those skilled in the art from the following detailed description, which shows and describes illustrative embodiments of the disclosed subject matter. Accordingly, the drawings and detailed description are to be regarded as illustrative in nature and not restrictive.

### BRIEF DESCRIPTION OF THE DRAWINGS

FIG. 1 is a schematic block diagram depicting an illustrative navigation system, in accordance with embodiments of the disclosed subject matter.

FIG. 2 is a schematic block diagram depicting an illustrative computing device, in accordance with embodiments of the disclosed subject matter.

FIG. 3 is a schematic block diagram depicting an exemplary demodulation unit, in accordance with embodiments of the disclosed subject matter.

FIG. 4 is a schematic block diagram depicting an exemplary multiplier, in accordance with embodiments of the disclosed subject matter.

FIG. 5 is a flow diagram depicting an illustrative method of an exemplary demodulation process of a demodulation unit using an electromagnetic navigation system, in accordance with embodiments of the disclosed subject matter.

While the disclosed subject matter is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

As the terms are used herein with respect to ranges of measurements (such as those disclosed immediately above), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

Although the term “block” may be used herein to connote different elements illustratively employed, the term should not be interpreted as implying any requirement of, or particular order among or between, various blocks disclosed herein. Similarly, although illustrative methods may be represented by one or more drawings (e.g., flow diagrams, communication flows, etc.), the drawings should not be interpreted as implying any requirement of, or particular order among or between, various steps disclosed herein. However, certain embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

### DETAILED DESCRIPTION

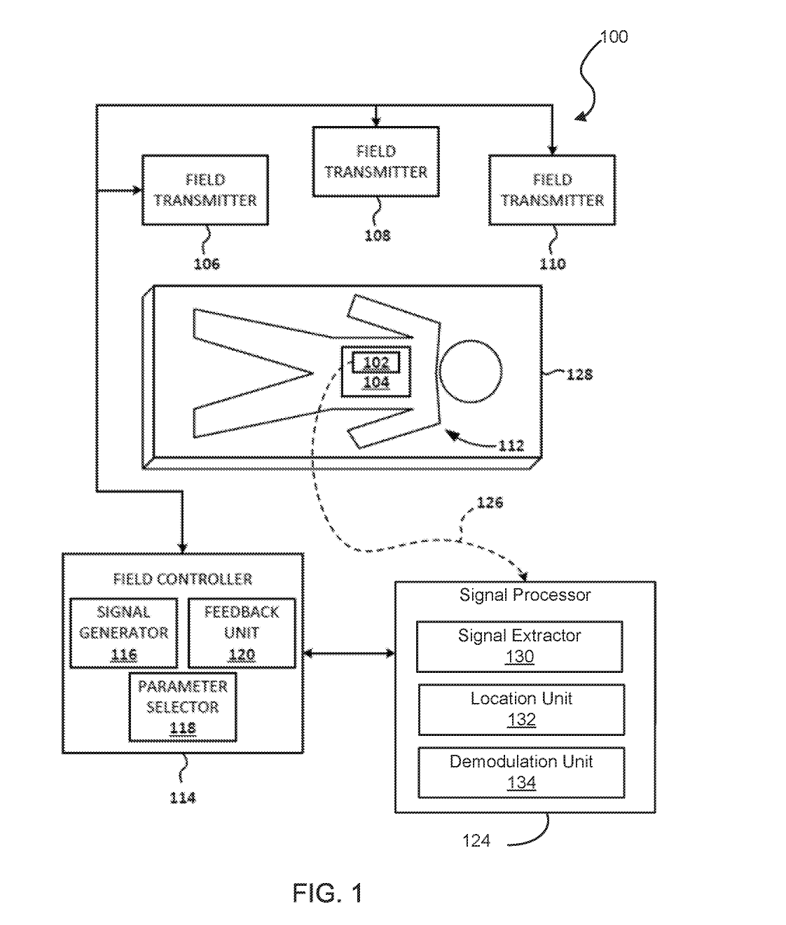

FIG. 1 is a schematic block diagram depicting an illustrative electromagnetic navigation system 100, in accordance with embodiments of the disclosed subject matter. The navigation system 100 is configured to determine, based on information collected using a receiver (or sensor) 102 associated with a medical device 104, location information corresponding to the medical device 104. The information collected by the receiver 102 includes a received field signal corresponding to an electromagnetic field defined by a set of electromagnetic signals transmitted by field transmitters 106, 108, and 110. A field transmitter may include, for example, a spool, a coil, a trace, and/or the like. According to embodiments, two or more field transmitters 106, 108, and 110, are configured to transmit (e.g., radiate) sinusoidal electromagnetic signals, which produce a field within which a subject 112 is disposed. According to embodiments, the system 100 includes a field controller 114 configured to manage operation of the transmitters 106, 108, and 110.

As shown in FIG. 1, the field controller 114 includes a signal generator 116 configured to provide a driving current to each of the field transmitters 106, 108, and 110, thereby causing each field transmitter to transmit an electromagnetic signal based on the driven current. In embodiments, the signal generator 116 may be configured to provide sinusoidal driving currents to the field transmitters 106, 108, and 110. Various parameter values of the driving currents may be configured based on receiving the parameter values from a parameter selector 118.

The parameter selector 118 may be configured to select one or more parameter values corresponding to one or more of the electromagnetic signals to be transmitted by one or more of the field transmitters 106, 108, and 110. The selected parameter values may include values of parameters such as, for example, frequency, amplitude, wavelength, period, phase, power, and/or the like. In embodiments, the parameter selector 118 selects a parameter value by determining the value of the parameter that satisfies a specified relationship. For example, in embodiments, the parameter selector 118 may be configured to evaluate a system of equations to solve for an independent variable, where the solution includes a parameter value. In this manner, for example, each field transmitter may be configured to emit a sinusoidal signal having a weighted mixture of frequencies, with the respective amplitudes (e.g., weights) obtained from solving a set of equations designed to suppress error terms of increasing order in frequency. The set of transmission frequencies is unique for each transmitter coil, and the relative amplitudes for each coil may be monitored and controlled within a pre-defined range.

According to embodiments, the field controller 114 may include a feedback unit 120 that is configured to receive feedback information from the field transmitters 106, 108, and 110, the receiver 102, and/or a signal processor 124 to determine whether to adjust the transmitted electromagnetic signals. That is, for example, the field controller 114 may be configured to determine whether any number of different types of criteria are satisfied and, based on that determination, to cause the electromagnetic signals to be adjusted. The feedback unit 120 may be configured to cause an electromagnetic signal to be adjusted by providing a control signal to the signal generator 116 to cause the signal generator 116 to modify the driving current that it provides to the corresponding field transmitter, and/or by providing a control signal to the parameter selector 118 to cause the parameter selector 118 to modify one or more determined parameter values before the parameter selector 118 provides the one or more parameter to the signal generator 116.

In this manner, embodiments may include a closed feedback loop that facilitates dynamically generating an electromagnetic field that satisfies any number of various types of criteria. For example, the feedback unit 120 may be configured to detect the occurrence of drift (e.g., due to heating of transmitter components), and may calculate an adjustment to adjust for the drift. In embodiments, for example, the feedback unit 120 may be configured to control the amplitudes of the signals (e.g., the weights associated with the frequencies) so as to maintain a particular relationship between the amplitudes. For example, in embodiments, the feedback unit 120 is configured to maintain the relationship between the multiple transmitted amplitudes to be within 1% of the relationship between the selected amplitudes.

The receiver 102 (which may include one or more receivers) may be configured to produce an electrical response to the field—referred to herein as a received field signal. That is, for example, the receiver 102 may include any magnetic field sensor, whether now known or later developed, including include sensors such as inductive sensing coils and/or various sensing elements such as magneto-resistive (MR) sensing elements (e.g., anisotropic magneto-resistive (AMR) sensing elements, giant magneto-resistive (GMR) sensing elements, tunneling magneto-resistive (TMR) sensing elements, Hall effect sensing elements, colossal magneto-resistive (CMR) sensing elements, extraordinary magneto-resistive (EMR) sensing elements, spin Hall sensing elements, and the like), giant magneto-impedance (GMI) sensing elements, and/or flux-gate sensing elements. The received field signal may include multiple received field signals, each of which may be processed to extract field components corresponding to one or more transmitters. The received field signal is communicated to a signal processor 124, which is configured to analyze the received field signal to determine location information corresponding to the receiver 102 (and, thus, the medical device 104). Location information may include any type of information associated with a location and/or position of a medical device 104 such as, for example, location, relative location (e.g., location relative to another device and/or location), position, orientation, velocity, acceleration, and/or the like. An exemplary signal processor 124 is described in commonly assigned U.S. Patent Application No. 62/436,411 filed Dec. 19, 2016.

The medical device 104 may include, for example, a catheter (e.g., a mapping catheter, an ablation catheter, a diagnostic catheter, introducer, etc.), an endoscopic probe or cannula, an implantable medical device (e.g., a control device, a monitoring device, a pacemaker, an implantable cardioverter defibrillator (ICD), a cardiac resynchronization therapy (CRT) device, a CRT-D device, etc.), and/or the like. For example, in embodiments, the medical device 104 may include a mapping catheter associated with an anatomical mapping system. The medical device 104 may include any other type of device configured to be at least temporarily disposed within a subject 112. The subject 112 may be a human, a dog, a pig, and/or any other animal having physiological parameters that can be recorded. For example, in embodiments, the subject 112 may be a human patient.

As shown in FIG. 1, the medical device 104 may be configured to be disposed within the body of a subject 112, and may be configured to be communicatively coupled to the signal processor 124 via a communication link 126 (shown in phantom). In embodiments, the communication link 126 may be, or include, a wired communication link (e.g., a serial communication), a wireless communication link such as, for example, a short-range radio link, such as Bluetooth, IEEE 802.11, a proprietary wireless protocol, and/or the like. The term “communication link” may refer to an ability to communicate some type of information in at least one direction between at least two devices, and should not be understood to be limited to a direct, persistent, or otherwise limited communication channel. That is, according to embodiments, the communication link 126 may be a persistent communication link, an intermittent communication link, an ad-hoc communication link, and/or the like. The communication link 126 may refer to direct communications between the medical device 104 and the signal processor 124, and/or indirect communications that travel between the medical device 104 and the signal processor 124 via at least one other device (e.g., a repeater, router, hub, and/or the like). The communication link 126 may facilitate uni-directional and/or bi-directional communication between the medical device 104 and the signal processor 124. Data and/or control signals may be transmitted between the medical device 104 and the signal processor 124 to coordinate the functions of the medical device 104 and/or the signal processor 124.

In operation, the time-varying electromagnetic field produced using the transmitters 106, 108, and 110 may be distorted or interfered by the presence of conductors within the environment. For example, as shown in FIG. 1, the subject 112 may be positioned on a conductive platform 128 (e.g., a metal bed), the room in which the subject 112 is disposed may include conductive medical equipment, structures, and/or the like. It will be understood by those having skill in the relevant arts that a time-varying electromagnetic field may produce eddy currents within a conductor, which may have distortive effects on the resulting electromagnetic field that is detected by the receiver 102.

As shown in FIG. 1, the signal processor 124 includes a signal extractor 130, which may be configured to extract, from the received field signal, signal components associated with each of the field transmitters 106, 108, and 110 (e.g., by deconvolving the received signal). Throughout this document, the term “deconvolve” and its derivatives (e.g., deconvolving, deconvolved, etc.) is used herein to refer to a process of “deconvolution,” refers to an operation that reverses effects of convolution. In this context, “deconvolving” and its derivatives are interchangeable with the term “deconvolute” and its derivatives (e.g., deconvoluting, deconvoluted, etc.) to the extent that those terms also refer to a process of deconvolution. The signal processor 124 further includes a location unit 132 configured to determine, based on the extracted signal components, location information corresponding to the medical device 104. The location unit 132 may be configured to determine location information according to any location-determination technique that uses magnetic navigation.

Also included in the signal processor 124 is a demodulation unit 134 configured to extract a desired signal from a plurality of received field signals corresponding to each transmitter 106, 108, and 110. In operation, the demodulation unit 134 extracts the desired signal at each frequency associated with a corresponding field transmitter 106, 108, 110 using a synchronization signal including at least one of a clock signal, a sine signal, and a cosine signal. Other suitable synchronization signals are also contemplated to suit different applications. For example, the received field signal is multiplied by the clock signal, having an alternating value between +1 and −1, of the same frequency of the corresponding field transmitter 106, 108, and 110 using a quadrature demodulation technique to reduce computational complexity.

Further, the demodulation unit 134 is configured to select a field transmitter frequency that are orthogonal (90°) using a frequency-division multiplexing scheme, such as an Orthogonal Frequency-Division Multiplexing (OFDM) technique. For example, the received field signal is further multiplied by the clock signal that is orthogonal, and the received field signal is heterodyned with the sine or cosine signal. Transmission frequencies of the field transmitters 106, 108, 110 are selected such that heterodyne frequencies of the received field signals are orthogonal over a predetermined time period. In this way, when the received field signal is further multiplied by the clock signal that is orthogonal, a sum of integral values of the clock signal and other undesired or unselected field transmitter frequencies during the predetermined time period becomes zero. Thus, only the desired signal from the plurality of received field signals is extracted, and the rest of the received field signals are suppressed.

According to various embodiments of the disclosed subject matter, any number of the components depicted in FIG. 1 (e.g., the receiver 102, the medical device 104, the field controller 114, and/or the signal processor 124) may be implemented on one or more computing devices, either as a single unit or a combination of multiple devices.

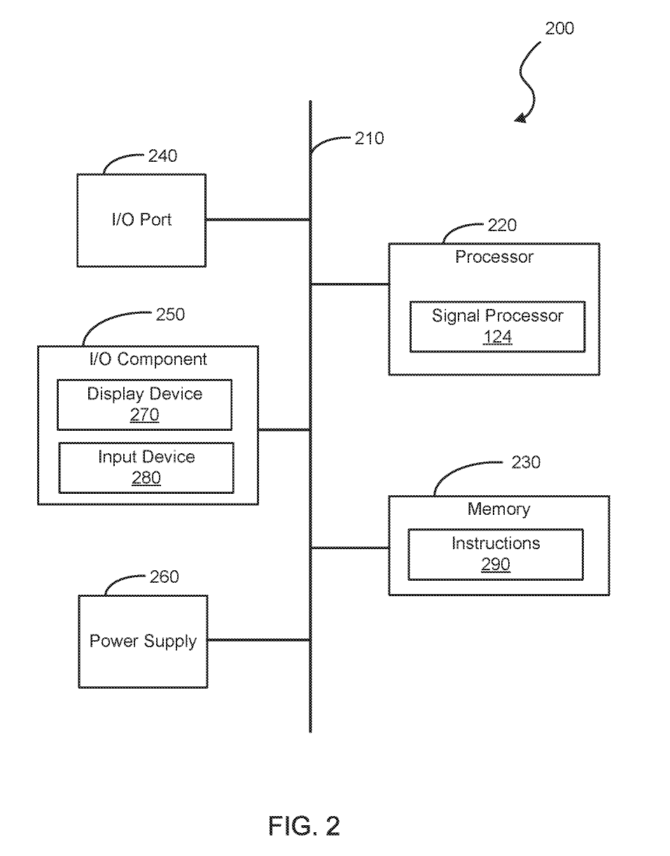

FIG. 2 is a schematic block diagram depicting an illustrative computing device 200, in accordance with embodiments of the disclosure. The computing device 200 may include any type of computing device suitable for implementing aspects of embodiments of the disclosed subject matter. Examples of computing devices include specialized computing devices or general-purpose computing devices such “workstations,” “servers,” “laptops,” “desktops,” “tablet computers,” “hand-held devices,” “general-purpose graphics processing units (GPGPUs),” and the like, all of which are contemplated within the scope of FIGS. 1 and 2, with reference to various components of the system 100 and/or computing device 200.

In embodiments, the computing device 200 includes a bus 210 that, directly and/or indirectly, couples the following devices: a processor 220, a memory 230, an input/output (I/O) port 240, an I/O component 250, and a power supply 260. Any number of additional components, different components, and/or combinations of components may also be included in the computing device 200. The I/O component 250 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The bus 210 represents what may be one or more busses (such as, for example, an address bus, data bus, or combination thereof). Similarly, in embodiments, the computing device 200 may include a number of processors 220, a number of memory components 230, a number of I/O ports 240, a number of I/O components 250, and/or a number of power supplies 260. Additionally any number of these components, or combinations thereof, may be distributed and/or duplicated across a number of computing devices. As an example only, the processor 220 may include the signal processor 124, but other suitable configurations are also contemplated to suit different applications.

In embodiments, the memory 230 includes computer-readable media in the form of volatile and/or nonvolatile memory and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In embodiments, the memory 230 stores computer-executable instructions 270 for causing the processor 220 to implement aspects of embodiments of system components discussed herein and/or to perform aspects of embodiments of methods and procedures discussed herein.

The computer-executable instructions 270 may include, for example, computer code, machine-useable instructions, and the like such as, for example, program components capable of being executed by one or more processors 220 associated with the computing device 200. Program components may be programmed using any number of different programming environments, including various languages, development kits, frameworks, and/or the like. Some or all of the functionality contemplated herein may also, or alternatively, be implemented in hardware and/or firmware.

The illustrative computing device 200 shown in FIG. 2 is not intended to suggest any limitation as to the scope of use or functionality of embodiments of the present disclosure. Neither should the illustrative computing device 200 be interpreted as having any dependency or requirement related to any single component or combination of components illustrated therein. Additionally, various components depicted in FIG. 2 may be, in embodiments, integrated with various ones of the other components depicted therein (and/or components not illustrated), all of which are considered to be within the ambit of the present disclosure.

Embodiments of an electromagnetic navigation system have been described herein, in which each of a number of field transmitters transmits a number of electromagnetic signals, each signal having a different frequency, with the relative proportions of individual frequencies in the combination of frequencies being selected to reduce a distortion component of the field signal received by a receiver.

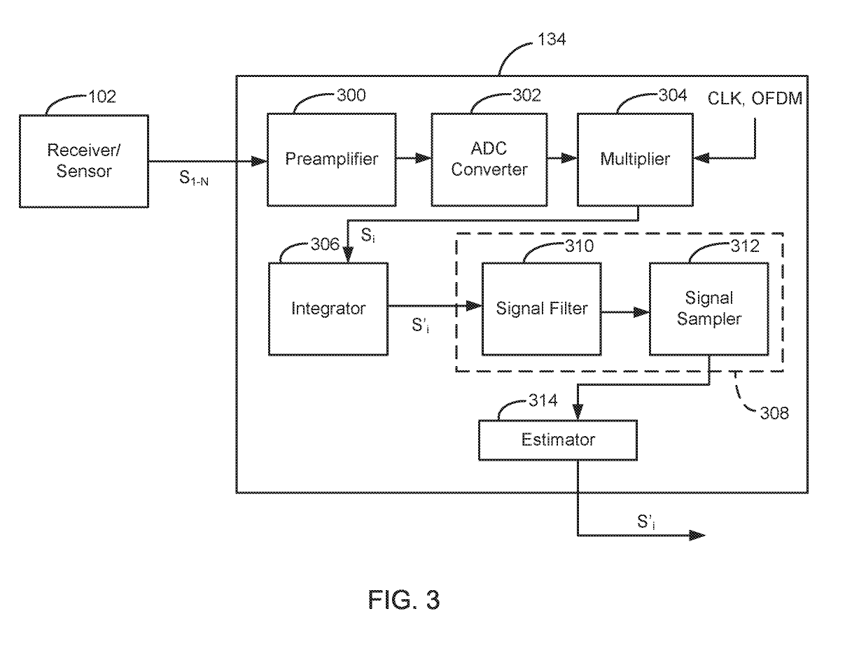

FIG. 3 is a schematic block diagram depicting an exemplary demodulation unit 134, in accordance with embodiments of the disclosed subject matter. In embodiments, an incoming field signal S_{1-N} from the receiver 102 may include a combination of the received field signals from the field transmitters 106, 108, 110, where N denotes a number of field transmitters (e.g., N=3 as shown in FIG. 1). In this example, the incoming field signal S₁₋₃ may be defined by expression (1):

S₁₋₃(t)=a₁ω₁+a₂ω₂t+a₃ω₃t (1)

where a₁, a₂, and a₃ denote amplitudes for three frequencies ω₁, ω₂, and ω₃, corresponding to each of the field transmitters 106, 108, 110, respectively, during a predetermined time period t.

Included in the demodulation unit 134 is a preamplifier 300 configured to amplify the incoming field signal S₁₋₃ and convert it into an amplified signal for subsequent processing. To reduce distortive effects of noise and interference, the preamplifier 300 may be disposed close to the receiver 102. Alternatively or additionally, an anti-aliasing filter may be used to restrict a bandwidth of the incoming field signal S₁₋₃ to satisfy a predetermined sampling theorem (e.g., Nyquist sampling theorem) over a frequency band of interest for filtering out unwanted signals. An analog-to-digital converter (ADC) 302 is also included in the demodulation unit 134 and is configured to convert the amplified or filtered incoming field signal S₁₋₃ into a digital signal.

A multiplier 304 of the demodulation unit 134 is configured to selectively extract a desired signal having a predetermined frequency from the received field signal based on a synchronization signal and an orthogonal synchronization signal. Specifically, undesired signals are suppressed from the received field signal based on the synchronization signal CLK (e.g., a clock signal) using the frequency-division multiplexing scheme (e.g., OFDM). As an example only, the desired signal Sᵢ may be extracted from the incoming field signal S₁₋₃, where i denotes an i^{th} signal having a selected frequency of the received field signal. An exemplary synchronization signal is a periodic signal with a predetermined harmonic frequency (e.g., a square wave).

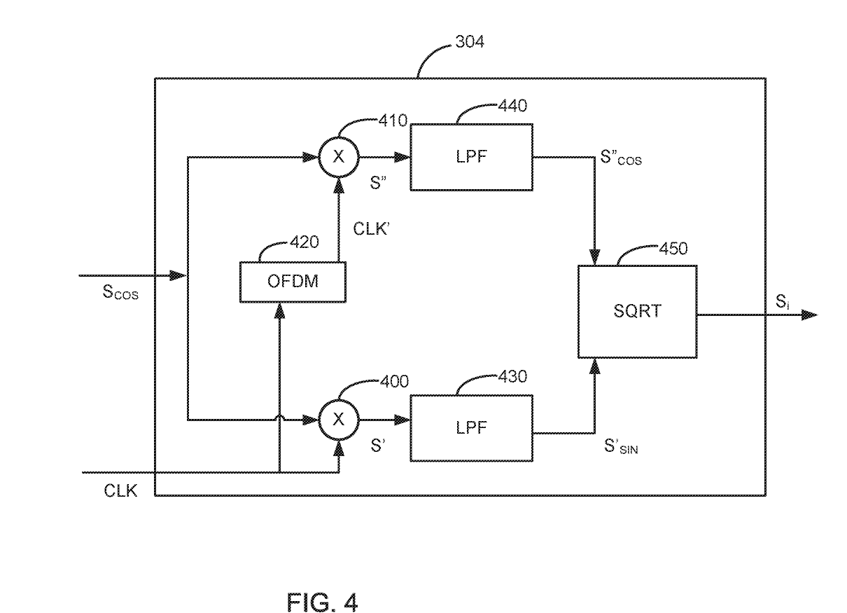

FIG. 4 is a schematic block diagram depicting an exemplary multiplier 304, in accordance with embodiments of the disclosed subject matter. In embodiments, the incoming field signal S₁₋₃ is initially heterodyned with a cosine signal. An exemplary incoming field signal S_{COS} may be defined by expression (2):

S_{COS}(t)=Σᵢ^{N}aᵢ cos ωᵢt (2)

where N=3 in this example, a₁, a₂, and a₃ denote amplitudes for three frequencies ω₁, ω₂, and ω₃, corresponding to each of the field transmitters 106, 108, 110, respectively, during a predetermined time period t.

A first mixer 400 is configured to multiply the incoming field signal S_{COS} by the synchronization signal CLK with the same frequency of the desired signal to be selected, and is also configured to generate a first mixed signal S′ based on the synchronization signal CLK and the incoming field signal S_{COS}. A second mixer 410 is configured to multiply the incoming field signal S_{COS} by the synchronization signal CLK′ that is orthogonal, which is generated based on a frequency-division multiplexing parameter 420 using the frequency-division multiplexing scheme (e.g., OFDM). As with the first mixer 400, the second mixer 410 is configured to generate a second mixed signal S″ based on the synchronization signal CLK′ that is orthogonal and the incoming field signal S_{COS}.

A first low pass filter 430 is configured to filter the first mixed signal S′ based on a first predetermined cutoff frequency to attenuate the first mixed signal with frequencies higher than the first predetermined cutoff frequency. In embodiments, the first mixed signal S′ is heterodyned with a sine signal to generate a first heterodyne signal S′_{SIN}. A second low pass filter 440 is configured to filter the second mixed signal S″ based on a second predetermined cutoff frequency to attenuate the second mixed signal with frequencies higher than the second predetermined cutoff frequency. In embodiments, the second mixed signal S″ is heterodyned with a cosine signal to generate a second heterodyne signal S″_{COS}. Sᵢ is the desired signal. In embodiments, the first and second low pass filters 430, 440 may be replaced by an integrator 306 performing similar operations of the filters.

Returning now to FIG. 3, the integrator 306 is configured to generate an integrated signal representing an integral value of the incoming field signal Sᵢ during a predetermined time period. As discussed above, the transmission frequencies of the incoming field signal Sᵢ are selected such that heterodyne frequencies of the incoming field signal Sᵢ are orthogonal over the predetermined time period. An exemplary integrated signal S_{INT} may be defined by expression (3):

$${S}_{INT} ( t ) = {\int }_{0}^{Tm} {\sum }_{i}^{N} \frac{{a}_{i}}{2} ( cos ( {ω}_{j} - {ω}_{i} ) + cos ( {ω}_{j} + {ω}_{i} ) ) \qquad ( 3 )$$

where Tₘ denotes a predetermined sampling interval (e.g., 25 milliseconds with a measurement frequency of 40 hertz (Hz)) with N=3 in this example, a₁, a₂, and a₃ denote amplitudes for three frequencies ω₁, ω₂, and ω₃, corresponding to each of the field transmitters 106, 108, 110, respectively.

Due to the selection of orthogonal frequencies over the predetermined sampling interval Tₘ, the field signal with the same frequency of the desired signal is selected, and the sum of the integral values of the clock signal and other undesired or unselected field signals having different frequencies becomes zero. An exemplary output signal S′ᵢ generated by the integrator 306 may be defined by expression (4):

$${S}_{i}^{′} ( t ) = \{ \begin{matrix}\frac{{a}_{i}}{2} Tm , j = i \\ 0 , j \neq 1\end{matrix} \qquad ( 4 )$$

where Tₘ denotes the predetermined sampling interval, a₁, a₂, and a₃ denote amplitudes for three frequencies ω₁, ω₂, and ω₃, corresponding to each of the field transmitters 106, 108, 110, respectively. As such, only the desired signal from the plurality of received field signals is extracted, and the rest of the received field signals are suppressed.

A decimator 308 of the demodulation unit 134 may include a signal filter 310 (e.g., an anti-aliasing or distortion filter) and a signal sampler 312 (e.g., a downsampling unit), and is configured to reduce a sampling rate of the output signal S′ᵢ for mitigating aliasing distortion. In embodiments, the signal filter 310 may be configured to restrict the bandwidth of the output signal S′ᵢ to satisfy the predetermined sampling theorem (e.g., Nyquist sampling theorem) over the frequency band of interest. In embodiments, the signal sampler 312 may be configured to downsample the filtered output signal S′ᵢ by a predetermined integer factor M such that only every M^{th} sample is processed. The signal filter 310 and the signal sampler 312 may be separate, independent units, or alternatively be combined as a single unit as the decimator 308 (shown in phantom). An estimator 314 of the demodulation unit 134 may be configured to remove or attenuate electrical crosstalk, such as electromagnetic interference gained during the predetermined sampling interval, from the output signal S′ᵢ. A corrected output signal S′ᵢ is outputted for subsequent processing.

As shown in FIG. 1, in embodiments, the parameter selector 118 may be configured to select as many frequencies as desired to suppress unwanted signals. The parameter selector 118 and feedback unit 120 can be integrated in embodiments, and may act as a control loop that serves to maintain the relative amplitudes of the transmitted mixture of frequencies. The control feedback loop may employ principles of control theory as well known and practiced in the art, such as Proportional-Integral-Derivative (PID) control, closed-loop transfer functions, and/or the like. In embodiments, the control feedback loop may employ one or more nonlinear control methods.

For example, the parameter selector 118 may select the frequencies ω₁=800 Hz, ω₂=1600 Hz, and ω₃=2400 Hz, and the parameter selector may determine that a₁=1, a₂=−1, and a₃=⅓. Thus, a first field transmitter in this example may be configured to transmit a signal at 800 Hz, 1600 Hz, and 2400 Hz. Different field transmitters transmit different sets of frequencies, with each frequency being uniquely used. In embodiments, for each set of frequencies, the frequencies may be configured to be within two to three bandwidths of one another. In this example, and for purposes of non-limiting illustration only, the bandwidth of the system may be in the range of at least approximately 40 Hz.

Using this bandwidth in the example discussed immediately above, a second transmitter may be configured to transmit a signal at 880 Hz, 1680 Hz, and 2480 Hz; and a third transmitter may be configured to transmit a signal at 960 Hz, 1760 Hz, and 2560 Hz. Thus, each transmitter may be configured to transmit a distinct set of frequencies, with no two frequencies (in the entire system) being identical. Furthermore, the frequencies may be separated by at least two system bandwidths (e.g., 2×40 Hz=80 Hz, in this example). The selected frequencies may be any frequency in the range that can be useful for electromagnetic navigation of an object such as a medical device, subject to the constraints of unique frequencies and sufficient separation as described above. In embodiments, for example, the frequencies may be greater than at least approximately 300 Hz and less than at least approximately 12,000 Hz.

The illustrative electromagnetic navigation system 100 shown in FIGS. 1-4 is not intended to suggest any limitation as to the scope of use or functionality of embodiments of the present disclosure. Neither should the illustrative system 100 be interpreted as having any dependency or requirement related to any single component or combination of components illustrated therein. Additionally, various components depicted in FIGS. 1-4 may be, in embodiments, integrated with various ones of the other components depicted therein (and/or components not illustrated), all of which are considered to be within the ambit of the present disclosure. For example, the field controller 114 and signal processor 124 may be implemented on a common computing device, using a common software module, and/or the like. As another example, in embodiments, any one or more of the signal generator 116, the parameter selector 118, and the feedback unit 120 may be integrated into one or more program or hardware components. Similarly, the signal extractor 130 and/or the location unit 132 and/or the demodulation unit 134 may be integrated into one or more program or hardware components.

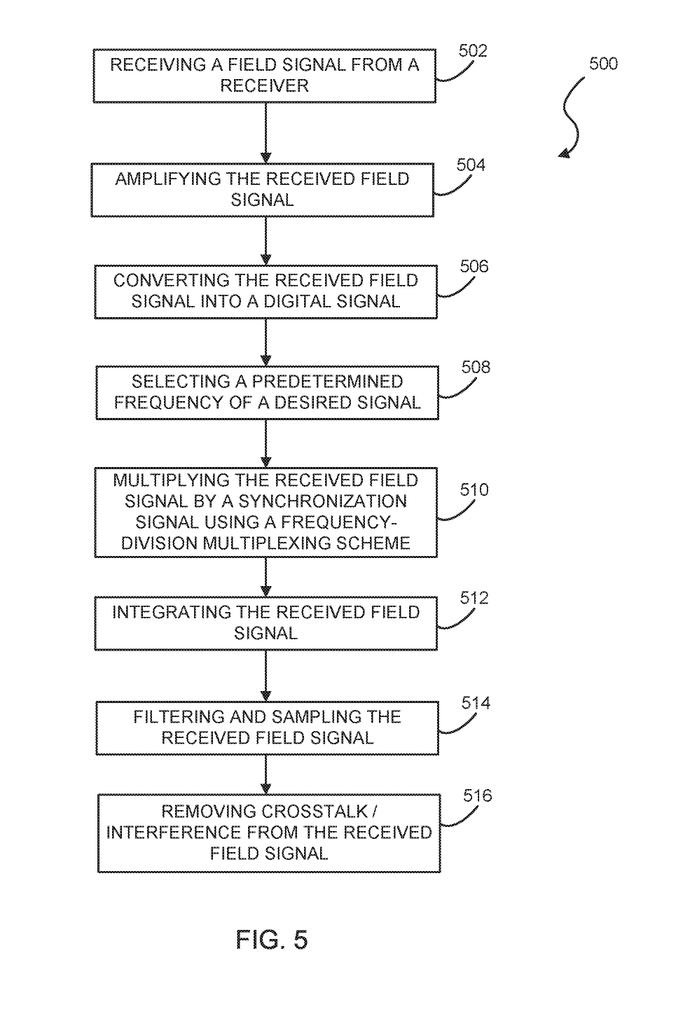

FIG. 5 is a flow diagram depicting an illustrative method 500 of an exemplary demodulation process of a demodulation unit using an electromagnetic navigation system for extracting a desired signal from a plurality of field signals, in accordance with embodiments of the disclosed subject matter. Embodiments of the method 500 may be performed by one or more components of an electromagnetic navigation system such as, for example, the navigation system 100 depicted in FIG. 1. In embodiments, the electromagnetic navigation system may include two or more field transmitters and at least one receiver. For example, the system may include between two and ten field transmitters. In embodiments, the system may include at least three field transmitters 106, 108, and 110, each of which transmits electromagnetic signals with at least three different frequencies.

As shown in FIG. 5, embodiments of the method 500 include receiving a field signal from a receiver, wherein the field signal includes a plurality of different frequencies corresponding to each of a plurality of field transmitters (block 502). As discussed above, sets of electromagnetic signals are transmitted using field transmitters 106, 108, and 110. Each electromagnetic signal of a set of electromagnetic signals being transmitted by the field transmitters 106, 108, and 110 may include a frequency that is different than a frequency associated with each of the other electromagnetic signals of the set of electromagnetic signals. In embodiments, each set of electromagnetic signals is a combination of sinusoids corresponding to a sum of individual sinusoidal functions, where each of the individual sinusoidal functions includes an amplitude and a frequency.

For example, embodiments may include transmitting, using a first field transmitter, a first set of electromagnetic signals, each electromagnetic signal of the first set of electromagnetic signals having a frequency that is different than a frequency associated with each of the other electromagnetic signals of the first set of electromagnetic signals, where each electromagnetic signal of the first set of electromagnetic signals is a sinusoid including an amplitude and a frequency, with the combined first set of electromagnetic signals corresponding to a first sum of these sinusoidal functions.

Similarly, embodiments may include transmitting, using a second field transmitter, a second set of electromagnetic signals, each electromagnetic signal of the second set of electromagnetic signals having a frequency that is different than a frequency associated with each of the other electromagnetic signals of the second set of electromagnetic signals, where each electromagnetic signal of the second set of electromagnetic signals is a sinusoid including an amplitude and a frequency, with the combined second set of electromagnetic signals corresponding to a second sum of these sinusoidal functions. In embodiments, each electromagnetic signal of the second set of electromagnetic signals may include a frequency that is different than a frequency associated with each of the electromagnetic signals of the first set of electromagnetic signals.

As shown in FIG. 5, in embodiments, the received field signal having the plurality of frequencies is amplified and converted into an amplified field signal (block 504). The amplified field signal is then further converted into a digital field signal using an ADC converter (block 506). When a predetermined frequency of a desired signal present in the received field signal is selected (block 508), the digital field signal is multiplied by the synchronization signal using the frequency-division multiplexing scheme, such as the OFDM technique (block 510). Specifically, the digital field signal is multiplied by the synchronization signal with the same frequency of the desired signal, and is converted into a first mixed signal. Similarly, the digital field signal is multiplied by the synchronization signal that is orthogonal and has the same frequency of the desired signal, and is converted into a second mixed signal.

In embodiments, the first mixed signal and the second mixed signal are integrated over a predetermined sampling interval and converted into an integrated signal having only the desired signal with the selected predetermined frequency (block 512). Thus, the received field signals having unselected frequencies are suppressed. Subsequently, the integrated signal is filtered to restrict its bandwidth over the frequency band of interest and is downsampled by a predetermined integer factor (block 514). The filtered and downsampled field signal is corrected by removing the electrical crosstalk or electromagnetic interference from the received field signal, and outputted for subsequent processing of the electromagnetic navigation system (block 516).

In embodiments, blocks 502 to 516 of the demodulation process are carried out continuously and repetitively over a succession of time steps that collectively define a larger time interval. In embodiments, the larger time interval may correspond to at least a portion of a medical procedure.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A method for using an electromagnetic navigation system having a plurality of field transmitters and a receiver, the method comprising:
receiving a field signal from the a receiver, the field signal having a plurality of different frequencies each corresponding to one of the plurality of field transmitters;
multiplying the received field signal by a synchronization signal using a frequency-division multiplexing scheme;
integrating the received field signal over a predetermined sampling interval and converting the received field signal into an integrated signal having only a desired signal based on the synchronization signal; and
outputting the integrated signal for subsequent processing of the electromagnetic navigation system.

2. The method of claim 1, further comprising suppressing unwanted signals having unselected frequencies from the received field signal.

3. The method of claim 2, further comprising amplifying the received field signal and converting the received field signal into a digital field signal.

4. The method of claim 3, further comprising selecting a predetermined frequency of the desired signal present in the received field signal.

5. The method of claim 4, further comprising filtering the received field signal by restricting a bandwidth of the received field signal.

6. The method of claim 5, further comprising downsampling the filtered received field signal by a predetermined integer factor.

7. The method of claim 6, further comprising correcting the received field signal by removing electrical crosstalk gained during the predetermined sampling interval from the received field signal.

8. The method of claim 7, further comprising:
generating a first mixed signal based on the synchronization signal and the received field signal; and
generating a second mixed signal based on the synchronization signal that is orthogonal and the received field signal.

9. The method of claim 8, further comprising extracting only the desired signal based on the first mixed signal and the second mixed signal.

10. The method of claim 8, further comprising filtering the first mixed signal based on a first predetermined cutoff frequency; and filtering the first mixed signal based on a second predetermined cutoff frequency.

11. The method of claim 1, wherein the synchronization signal is at least one of a clock signal, a sine signal, and a cosine signal.

12. The method of claim 1, wherein the received field signal is heterodyned with at least one of a sine signal and a cosine signal.

13. The method of claim 12, further comprising selecting transmission frequencies of the plurality of field transmitters such that heterodyne frequencies of the received field signals are orthogonal over a predetermined time period.

14. The method of claim 1, wherein the predetermined sampling interval is approximately 25 milliseconds with a measurement frequency of approximately 40 hertz.

15. The method of claim 1, further comprising generating a sum of zero integral value for unwanted signals having unselected frequencies from the received field signal based on the synchronization signal.

16. A method for using an electromagnetic navigation system having a plurality of field transmitters and a receiver, the method comprising:
receiving a field signal from the a receiver, the field signal having a plurality of different frequencies each corresponding to one of the plurality of field transmitters;
multiplying the received field signal by a synchronization signal using an Orthogonal Frequency-Division Multiplexing (OFDM) technique;
integrating the received field signal over a predetermined sampling interval and converting the received field signal into an integrated signal having only a desired signal based on the synchronization signal; and
outputting the integrated signal for subsequent processing of the electromagnetic navigation system.

17. The method of claim 16, further comprising filtering the received field signal by restricting a bandwidth of the received field signal.

18. The method of claim 17, further comprising downsampling the filtered received field signal by a predetermined integer factor.

19. An electromagnetic navigation system having a plurality of field transmitters and at least one receiver, the system comprising:
a receiver configured for receiving a field signal from the at least one receiver, the field signal having a plurality of different frequencies corresponding to each of the plurality of field transmitters;
a multiplier configured for selectively extracting a desired signal from the received field signal based on a synchronization signal and an orthogonal synchronization signal using a frequency-division multiplexing scheme;
an integrator configured for integrating the received field signal over a predetermined sampling interval and converting the received field signal into an integrated signal having only a desired signal based on the synchronization signal; and
an estimator configured for outputting the integrated signal for subsequent processing of the electromagnetic navigation system.

20. The system of claim 19, further comprising:
an amplifier configured for amplifying the received field signal;
a converter configured for converting the received field signal into a digital field signal;
a signal filter configured for filtering the received field signal by restricting a bandwidth of the received field signal; and
a signal sampler configured for downsampling the filtered received field signal by a predetermined integer factor.
